(19) United States
(12) Patent Application Publication
(10) Pub. No.: US 2024/0268943 A1
(43) Pub. Date: Aug. 15, 2024

(54) Dental Apparatus with Antimicrobial Activity

(71) Applicant: ZINKH NV, Gent (BE)

(72) Inventors: Shane DE VREESE, Gent (BE); Guy DE VREESE, Gent (BE)

(21) Appl. No.: 18/276,835

(22) PCT Filed: Feb. 11, 2022
(86) PCT No.: PCT/IB2022/051239

§ 371 Date: Aug. 10, 2023

(30) Foreign Application Priority Data
Feb. 12, 2021 (BE) 2021/5102

(51) Int. Cl. A61C 19/06; C08K 3/08

(52) U.S. Cl. CPC A61C 19/063; C08K 3/08; A61C 2201/00; C08K 2003/0893

(57) ABSTRACT

The present invention relates to a dental apparatus, wherein the apparatus is made of a polymer composition having antimicrobial activity, wherein the polymer composition contains one or more metal ions and/or metal salts at a concentration of between 0.001% and 10%. The present invention also relates to the use of said dental apparatus in the prevention or treatment of bacterial oral diseases, such as gingivitis, periodontitis and halitosis. As a final aspect, the present invention also relates to a method for producing said dental apparatus.

### TECHNICAL FIELD

The present invention relates to a dental apparatus made of a polymer composition having antimicrobial properties. Furthermore, the invention also relates to the use of this dental apparatus in the prevention or treatment of bacterial oral diseases. In a final aspect, the invention relates to a method of producing said dental apparatus.

### PRIOR ART

Different types of bacteria are present in the oral cavity. These bacteria usually live in an ecological balance and have more positive than negative effects on humans. In addition to the many good bacteria that have a protective function and help get digestion going, there are also pathogenic or disease-causing bacteria present. Those bacteria can attach themselves to the tooth enamel and form dental plaque. When dental plaque calcifies, we speak of tartar. When the balance is disturbed, this can lead to an excess of pathogenic bacteria. This in turn can lead to the development of bad breath (halitosis), but also to diseases, such as inflammation of the gums (gingivitis) or inflammation of the bone around the teeth (periodontitis). Today, about ⅔ of adults suffer from oral infections. This is worrying, especially since the majority of those people do brush their teeth on a regular basis and see a dentist regularly. Antibiotics temporarily eliminate the bacteria in the oral cavity, but over time (usually after a few days) the bacteria just return. In fact, because a lot of good bacteria were also removed by the antibiotic, the pathogenic bacteria get the chance to multiply undisturbed. Viral pathogens in the oral cavity also have a detrimental effect on the general health of humans.

There is thus a need for alternative solutions to prevent the multiplication of harmful pathogens in the oral cavity.

The control of oral pathogens by means of metal ions and/or metal salts has already been described several times. For example, there are currently toothpastes on the market with added metal ions and/or metal salts for antimicrobial activity.

Polymers with antimicrobial properties are also known; they are obtained by mixing the polymer with metal ions or metal salts, the antimicrobial and antiseptic properties being due to the controlled release of the ions from these metals.

For example, U.S. Pat. No. 7,661,430 describes a dental apparatus, mouthguard or other mouthpiece made of a polymer composition combined with metal particles or organic/inorganic metal complexes, wherein antimicrobial activity is obtained by the controlled release of the metals from the polymer composition.

U.S. Pat. No. 5,019,096, too, describes a method for making an infection-resistant medical device, wherein one or more polymers are combined with an antimicrobial component, namely a combination of silver salt and a biguanide. This antimicrobial component is released from the medical device in a controlled manner.

Although the intended result is achieved, which is to combat the proliferation of bacteria, the metal ions are released from the polymer over time, which can lead to toxic side effects.

The present invention aims to find a solution for at least some of the above problems.

### SUMMARY OF THE INVENTION

The invention relates to a dental apparatus according to claim 1. More particularly, the present invention discloses a dental apparatus, wherein the apparatus is made of a polymer composition having antimicrobial activity, wherein the polymer composition contains one or more metal ions and/or metal salts at a concentration of between 0.001% and 10%. Preferred embodiments of this dental apparatus are set forth in claims 2-8.

Because one or more metal ions and/or metal salts are contained in the polymer composition of the dental apparatus at a concentration between 0.001% and 10%, on the one hand antimicrobial activity is provided to the dental apparatus and on the other hand the mechanical properties of the polymer such as hardness, melting temperature and flexibility are preserved. However, this concentration has been found to be sufficient to be effective and capable of inhibiting the action of harmful pathogens, such as bacteria and viruses, in the oral cavity. In addition, this concentration is sufficiently low to minimize the toxicity of the metals.

In a second aspect, the invention relates to a use according to claim 9. More particularly, the present invention describes the use of said dental apparatus in the prevention or treatment of bacterial oral diseases, such as gingivitis, periodontitis and halitosis.

In a final aspect, the invention relates to a method according to claim 10. More particularly, the present invention describes a method for producing said dental apparatus, wherein one or more metal ions and/or metal salts are mixed with a polymer before the dental apparatus is formed by injection molding, extrusion, thermoforming, compression molding or 3D printing the mixture. Preferred embodiments of this method are set forth in claims 11-12.

### DETAILED DESCRIPTION

There is a need for a dental apparatus with antimicrobial properties, wherein any toxic side effects of the use of metal ions and/or metal salts is avoided. The present invention provides a solution for this.

Unless otherwise defined, all terms used in the description of the invention, including technical and scientific terms, have the meaning as commonly understood by a person skilled in the art to which the invention pertains. For a better understanding of the description of the invention, the following terms are explained explicitly.

In this document, “a” and “the” refer to both the singular and the plural, unless the context presupposes otherwise. For example, “a segment” means one or more segments.

When the term “around” or “about” is used in this document with a measurable quantity, a parameter, a duration or moment, and the like, then variations are meant of approx.

20% or less, preferably approx. 10% or less, more preferably approx. 5% or less, even more preferably approx. 1% or less, and even more preferably approx. 0.1% or less than and of the quoted value, insofar as such variations are applicable in the described invention. However, it must be understood that the value of a quantity used where the term ‘about’ or ‘around’ is used, is itself specifically disclosed.

The terms “comprise”, “comprising”, “consist of”, “consisting of”, “provided with”, “have”, “having”, “include”, “including”, “contain”, “containing” are synonyms and are inclusive or open terms that indicate the presence of what follows, and which do not exclude or prevent the presence of other components, characteristics, elements, members, steps, as known from or disclosed in the prior art.

“Antimicrobial activity” as described in this document refers on the one hand to the polymer's inhibitory effect on microbial growth in the oral cavity and, on the other hand, to the antiseptic effect of the polymer, preventing microbial contamination of the dental apparatus.

“Atomic absorption spectrometry (AAS)” as described in this document describes a collection of analytical techniques for quantitative determination of elements, including inductively coupled plasma atomic emission spectrometry (ICP-AES) and inductively coupled plasma mass spectrometry (ICP-MS).

Quoting numerical intervals by endpoints comprises all integers, fractions and/or real numbers between the endpoints, these endpoints included.

Bacteria are everywhere and not all bacteria are bad bacteria. The danger is mainly in certain specific pathogenic bacteria and especially when they grow too much and cause inflammation. Today, about ⅔ of adults suffer from oral infections. That is worrying, especially since the majority of those people do brush their teeth regularly and see a dentist on a regular basis. A dental apparatus with which people can counteract bacterial growth could limit the consequences of this bacterial growth. Antibiotics temporarily eliminate the bacteria in the oral cavity, but over time, the bacteria just return. In fact, because a lot of good bacteria were also removed by the antibiotic, the pathogenic bacteria get the chance to multiply undisturbed. Metal ions and/or the corresponding metal salts may possess antibacterial properties, for example by inhibiting certain bacterial enzymes important for the survival of the bacteria (bactericidal properties) or by reducing the formation of bacterial biofilms (bacteriostatic properties).

Dental apparatuses are inserted into the oral cavity and can sometimes be present there for long periods, such as nighttime braces worn during the night.

In a first aspect, the invention relates to a dental apparatus, wherein the apparatus is made of a polymer composition having antimicrobial activity, wherein the polymer composition contains one or more metal ions and/or metal salts at a concentration of between 0.001% and 10%, more preferably between 0.001% and 5%, more preferably between 0.001% and 3%, more preferably between 0.001% and 2%, more preferably between 0.001% and 1%, more preferably between 0.001% and 0.5%, more preferably between 0.01% and 0.1%.

The antimicrobial and antiseptic properties of the plastic composition are due to the presence of the ions of these metals. This is because metal ions show antimicrobial activity, including broad-spectrum bacteriostatic and bactericidal properties.

Because the dental apparatus is made of a polymer composition having antimicrobial activity, the dental apparatus will on the one hand be able to reduce the amount of bacteria and viruses present in the oral cavity. A standardized method for measuring antibacterial activity on plastics and other non-porous surfaces is described in ISO22196. In addition, the polymer composition will also exhibit antiseptic properties, limiting microbial growth and biofilm formation on the dental apparatus, thus limiting the risk of bacteria entering the oral cavity in this way. By using a polymer composition that is easily deformable (e.g., a thermoplastic), the dental apparatus can be easily adapted to the needs and wishes of the user.

Because one or more metal ions and/or metal salts are contained in the polymer composition of the dental apparatus at a concentration between 0.001% and 10%, on the one hand antimicrobial activity is provided to the dental apparatus and on the other hand the mechanical properties of the polymer composition such as hardness, melting temperature and flexibility are preserved. This concentration of metal ions and/or metal salts also has a very low toxicity, making the polymer composition extremely suitable for use in dental apparatuses.

In the context of the present invention, the term ‘dental apparatus’ is understood to mean any device that can be placed in the oral cavity of a user, which will perform a corrective, preventive and/or sterilizing function there. More specifically, in one embodiment, said dental apparatus will comprise orthodontic braces, a sports mouthguard, a gingivitis mouthguard, an orthodontic aligner, a temporary crown, a permanent crown, a bite plate, dentures, an antibacterial pad, a palatal splint, an anti-snoring device, a whitening shield, a bruxism splint, a coping, a fluoride shield, an individual impression tray, a Michigan splint, a medication shield, a radiation shielding splint, an occlusal splint, an interim prosthesis, or a similar device.

If the compatibility between the metal ions and/or metal salts and the polymer is insufficient, the polymer will release the metal ions over time. Thus, in a preferred embodiment, the metal ions and/or salts of said metal ions are dispersed or dissolved in the polymer. This is achieved by adding the metal ions and/or metal salts to a solution or a dispersed phase of monomers before or during the polymerization reaction of these monomers. In this way, the metal ions and/or metal salts will be incorporated into the plastic composition and the compatibility between the plastic and the metal ions and/or metal salts will be such that the released concentration of metal ions remains limited, even for extended periods.

Also when the polymer composition is used in the manufacture of equipment, such as a dental apparatus according to the present invention, the bond between the polymer and the metal salts will be such that the released concentration of metal ions remains limited. This is important, especially for equipment that comes into close contact with the body, such as the oral cavity. This is because the release of too high a concentration of metal ions can lead to undesirable side effects. This can range from irritation to chronic intoxication and can even lead to serious conditions such as neurological problems.

In one embodiment, the released concentration of the metal ions when using the apparatus in the oral cavity of a patient is up to 21 parts per million (ppm) more preferably between 0 and 5 ppm, more preferably between 0 and 1 ppm, more preferably between 0 and 0.5 ppm. Such exceptionally low to no release of metal ions can be described as “no leaching”.

In a preferred embodiment, no such values are exceeded when the apparatus is exposed for at least 24 hours, preferably at least 36 hours, to an aqueous environment, such as for example the oral cavity.

By limiting the release of the metal ions to this amount, the concentration of the metal ions in the oral cavity and in the rest of the body is limited. Excessively high concentrations of metal ions in the body can lead to certain symptoms. For example, the prolonged use of colloidal silver or silver particles can lead to argyria or skin necrosis. Chronic exposure to high doses of copper can in turn lead to liver cirrhosis, hemolysis, and damage to the kidneys, brain and other organs, which can lead to coma, necrosis of liver cells, circulatory failure and death. Acute zinc overdose causes mainly gastrointestinal symptoms. Chronic exposure to too high a dose of zinc has negative effects on cell metabolism in the body and the immune system. Furthermore, this increases the risk of prostate cancer and can affect the taste sensation.

Notwithstanding the limited release of the metal ions into the oral cavity, the inventors of the present invention found that the polymeric dental apparatus exhibits antimicrobial activity. Without wishing to be bound by a theory, this seems to be due in part to a positive influence of the metal ions present on the functioning of the salivary glands, thereby influencing microbial growth and the formation of a charged ion field that prevents pathogens such as bacteria and viruses from adhering to the polymer and nearby structures. By stopping microbial growth in the presence of disease, the immune system has a chance to regain control of the inflammation and heal the body.

The possible migration of the ions out of the plastic can be quantified by means of tests known for this purpose, such as migration tests. These are performed under established standard conditions. The polymer with the incorporated metal ions and/or metal salts is immersed in a known volume of a specific simulant and the migration of ions from the polymer to the simulant is determined. Examples of such simulants are water and BHI (Brain Heart Infusion) medium. Suitable analysis techniques for measuring the ion concentrations are on the one hand atomic spectrometric techniques, such as inductively coupled plasma atomic emission spectrometry (ICP-AES) and inductively coupled plasma mass spectrometry (ICP-MS) and on the other hand electrochemical analysis techniques such as stripping voltammetry. XPS (X-ray photoelectron spectrometry), XRF (X-ray fluorescence analysis), EDAX (electron dispersive X-ray analysis) and SIMS (secondary ion mass spectrometry) can also be used for measuring ion concentrations. In a preferred embodiment, ICP-AES is employed to determine the migration of ions from polymers.

In one embodiment, the metal ion is selected from the group consisting of Al (aluminum), Si (silicon), Ti (titanium), V (vanadium), Cr (chromium), Mn (manganese), Co (cobalt), Ni (nickel), Cu (copper), Zn (zinc), Zr (zirconium), Nb (niobium), Mo (molybdenum), Pd (palladium), Ag (silver), Sn (tin), Se (selenium), Ta (tantalum), W (tungsten), Pb (lead), Au (gold), Pt (platinum) or a combination of these.

These metal ions are known for their antimicrobial and antiseptic activity. In a preferred embodiment, the size of the ion particles is between 1 μm and 5 μm.

In a preferred embodiment, the metal ion is zinc and zinc is present in the polymer in the form of a zinc salt, preferably zinc pyrrolidone carboxylate (zinc PCA), zinc oxide, zinc hydroxide, zinc pyrrolidone, zinc pyrithione, a zinc salt of a fatty acid or a mixture thereof. In one embodiment, zinc is present in the form of a mixture of zinc salts of fatty acids.

Zinc is an essential trace element. Among other things, it plays an important role in energy production in the body, cell metabolism, DNA and RNA synthesis and regulation of the immune system. Furthermore, zinc is known for its antimicrobial properties. Zinc, for example, is present on the top layer of the skin and forms a defense barrier against viruses and bacteria. In recent years, numerous studies have confirmed the antibacterial and antiviral properties of zinc ions, especially against Gram⁺ bacteria such as streptococci and actinomycetes, important bacteria in the oral cavity. Indeed, zinc ions have bacteriostatic and bactericidal properties, which means that they can inhibit the growth of bacteria and block various biological processes that are fundamental for the survival of the bacteria themselves or for the formation of bacterial biofilms.

In one embodiment, the polymer composition consists of one or more polymers, wherein the polymer is selected from the group of acrylates and methacrylates (such as polymethyl acrylate (PMA), polymethyl methacrylate (PMMA)), polyesters (such as polyethylene terephthalate (PET), polyethylene terephthalate glycol (PETG), polycaprolactone (PCL), polysulfone (PSU), polylactic acid (PLA), poly(lactic-co-glycolic acid) (PLGA), polybutylene terephthalate (PBT), polyethylene furanoate (PEF), polypropylene terephthalate (PPT), polybutylene adipate terephthalate (PBAT), polybutylene succinate (PBS), polybutylene succinate adipate (PBSA)), thermoplastic polyurethanes (TPU), polyether blockamides (PEBAX), polyolefins and polyolefin copolymers (such as polyethylene (PE), polypropylene (PP), ethylene vinyl acetate (EVA), polyvinyl butyraldehyde (PVB), polybutylene (PB), polyisobutylene (PIB), ethylene propylene diene monomer (EPDM)), styrene-based thermoplastics (such as poly(styrene-butadiene-styrene) (SBS), polystyrene, acrylonitrile butadiene styrene (ABS), acrylonitrile styrene acrylate (ASA), and styrene acrylonitrile (SAN)), polyamides (such as polyamide 6, polyamide 6,6 or polyamide 12), polycarbonate (PC) and polyacetals (such as polyoxymethylene (POM)), silicones or a mixture thereof.

Poly(lactic-co-glycolic acid) (PLGA), for example starch- and sugar- and cellulose-based products and polyhydroxyalkanoates, has the advantage of being biodegradable and having a lower impact on the environment.

In a preferred embodiment, the polymer composition comprises a thermoplastic polymer. More than 80% of industrial polymers are thermoplastics. A thermoplastic is a plastic material that softens when heated. This is in contrast to thermosets, materials that remain hard when heated. An advantage of thermoplastic material is the possibility of reuse in various applications. Reheating allows the material to be shaped into other desired shapes. As already indicated, in the context of the present invention, it is particularly interesting to use a thermoplastic, so that the dental apparatus can be shaped in this way according to the wishes and needs of the user. Vitrimers, thermosets that can be deformed again when heated, are also within the scope of this invention.

Different polymers can be combined to obtain a polymer composition with the desired characteristics.

As already mentioned, due to the concentration of metal ions contained in the polymer composition, the dental apparatus according to the present invention has an antimicrobial activity. This allows the apparatus, when placed in the oral cavity, to effect a reduction in the amount of bacteria present there. Also, due to its antimicrobial effect, the device can protect an open wound, for example, and thereby accelerate and ensure the healing of this open wound. Furthermore, the concentration of metal ions contained in the polymer composition also provides an antiseptic effect, limiting among other things bacterial growth and biofilm formation on the dental apparatus. This offers advantages in the maintenance of the dental apparatus, preventing bacterial contamination of the dental apparatus. This minimizes bacterial proliferation in the oral cavity that could be caused by growth on the dental apparatus.

In that regard, in a second aspect, the invention relates to an aforementioned dental apparatus for use in the prevention or treatment of bacterial oral diseases, such as gingivitis, periodontitis and halitosis. Bacteria survive on waste products in the oral cavity, such as food debris, dead epithelial cells or components of the saliva. As is also the case with the human metabolism, this produces end products that have to be excreted. The anaerobic bacteria (which live without oxygen) produce, among other things, sulfur as an end product. That gas often causes bad-smelling breath. Various ions have been described in the literature that have a direct inhibitory effect on the volatilization of H2S and thereby contribute to the reduction of bad breath. Use of the dental apparatus according to the present invention, wherein such metal ions are contained in the polymer composition from which the dental apparatus is made, makes it possible to reduce bacterial growth and thus counteract the onset and/or progression of halitosis.

Gingivitis or inflammation of the gums is characterized by redness and swelling of the gums. The gums also bleed easily when touched. Gingivitis is caused by the buildup of dental plaque and tartar around the teeth. Tartar has a very rough surface to which bacteria can adhere more easily than on the tooth surface. If the inflammation involves more tissue than just the gums, it is called periodontitis. This lowers the bone level and creates deepened gum pockets (this is a space between the tooth and gums). In these gum pockets, anaerobic bacteria have a chance to multiply undisturbed. The inflammation can be caused by several bacteria, such as Porphyromonas gingivalis, Treponema denticola, Prevotella intermedia, Fusobacterium nucleatum and Eubacterium. Gingivitis and periodontitis can be cured by removal of dental plaque and tartar by the dentist, but also by eliminating bacterial growth in the oral cavity. Use of the dental apparatus according to the present invention makes it possible to reduce bacterial growth and thus counteract the onset and/or progression of gingivitis and periodontitis.

In a final aspect, the invention relates to a method for producing an aforementioned dental apparatus, wherein one or more metal ions and/or metal salts are mixed with a polymer before the dental apparatus is formed by injection molding, extrusion, thermoforming, compression molding or 3D printing the mixture.

In one embodiment, an active mixture of metal ions and/or metal salts is mixed into a polymer composition by compounding. “Compounding” is the creation of formulations for the production of plastics and/or synthetic fibers by mixing/blending polymers and additives in a molten state.

In an alternative embodiment, the method comprises:
dissolving or dispersing one or more metal ions or metal salts in an aqueous or organic solvent,
adding this obtained solution or dispersion of the metal ion and/or metal salt to a solution or a dispersed phase of monomers suitable for synthesizing a polymer by means of a polymerization reaction,
carrying out the polymerization reaction,
forming a dental apparatus by injection molding, extrusion, thermoforming, compression molding or 3D printing the polymerized composition.

In one embodiment, after adding the solution or dispersion of the metal ion and/or metal salt to a solution or dispersed phase of monomers, the whole is stirred until a polymer is obtained from the polymerization of the monomers.

In one embodiment, the metal ion is present in the form of a mixture of organic metal salts by (carboxyl) fatty acids.

In one embodiment, the metal ion is selected from the group consisting of Al (aluminum), Si (silicon), Ti (titanium), V (vanadium), Cr (chromium), Mn (manganese), Co (cobalt), Ni (nickel), Cu (copper), Zn (zinc), Zr (zirconium), Nb (niobium), Mo (molybdenum), Pd (palladium), Ag (silver), Sn (tin), Se (selenium), Ta (tantalum), W (tungsten), Pb (lead), Au (gold), or Pt (platinum). In a preferred embodiment, the metal ion is zinc and zinc is present in the polymer in the form of a zinc salt, preferably zinc pyrrolidone carboxylate (zinc PCA), zinc oxide, zinc hydroxide, zinc pyrrolidone, zinc pyrithione, a zinc salt of a fatty acid or a mixture thereof.

In one embodiment, the polymer composition consists of one or more polymers, wherein the polymer is selected from the group of acrylates and methacrylates (such as polymethyl acrylate (PMA), polymethyl methacrylate (PMMA)), polyesters (such as polyethylene terephthalate (PET), polyethylene terephthalate glycol (PETG), polycaprolactone (PCL), polysulfone (PSU), polylactic acid (PLA), poly(lactic-co-glycolic acid) (PLGA), polybutylene terephthalate (PBT), polyethylene furanoate (PEF), polypropylene terephthalate (PPT), polybutylene adipate terephthalate (PBAT), polybutylene succinate (PBS), polybutylene succinate adipate (PBSA)), thermoplastic polyurethanes (TPU), polyether blockamides (PEBAX), polyolefins and polyolefin copolymers (such as polyethylene (PE), polypropylene (PP), ethylene vinyl acetate (EVA), polyvinyl butyraldehyde (PVB), polybutylene (PB), polyisobutylene (PIB), ethylene propylene diene monomer (EPDM)), styrene-based thermoplastics (such as poly(styrene-butadiene-styrene) (SBS), polystyrene, acrylonitrile butadiene styrene (ABS), acrylonitrile styrene acrylate (ASA), and styrene acrylonitrile (SAN)), polyamides (such as polyamide 6, polyamide 6,6 or polyamide 12), polycarbonate (PC) and polyacetals (such as polyoxymethylene (POM)), silicones or a mixture thereof.

The solvent used in step a of the aforementioned method is preferably selected from: water, ethyl alcohol, methanol, acetone, isopropyl alcohol, ethyl acetate, acetonitrile, or a mixture of two or more solvents. In one embodiment, water is used in the mixture in a percentage between 5% and 100%, preferably between 20% and 100% by weight. In one embodiment, ethyl alcohol is used in the mixture in a percentage between 5% and 15%, preferably between 5% and 10% by weight. In one embodiment, methanol is used in the mixture at a percentage between 5% and 10%, preferably between 5% and 7% by weight. In one embodiment, acetone is used in the mixture in a percentage between 3% and 70%, preferably between 10% and 65% by weight. In one embodiment, isopropyl alcohol is used in the mixture in a percentage between 2% and 20%, preferably between 5% and 15% by weight.

In an embodiment of the method, the resulting solution or dispersion of the metal ion or metal salt is added to a solution or dispersed phase of monomers before the polymerization reaction. In another embodiment of the method, the resulting solution or dispersion of the metal ion or metal salt is added to a solution or dispersed phase of monomers during the polymerization reaction. In a preferred embodiment, the solution or dispersion of the metal ion or metal salt is added dropwise to the solution or dispersed phase of monomers. By adding the resulting solution or dispersion of the metal ion or metal salt to a solution or a dispersed phase of monomers added before or during the polymerization reaction, the metal ions and/or metal salts are incorporated into the polymer and the release of the metal ions and/or metal salts from the polymer is minimized.

The polymerization reaction can be carried out in any manner known in the art. In one embodiment, a suitable catalyst is used to achieve the desired stereoselectivity or tacticity.

Forming techniques for forming the dental apparatus include any technique known in the art, such as injection molding, extrusion, thermoforming, compression molding or 3D printing. With injection molding, plastic which is supplied as granulate or powder, is melted to a viscous mass and is injected under high pressure into a mold of which the cavity is the shape of the desired product. Cooling solidifies the plastic, and the desired product is obtained. Injection molding is one of the most commonly used forming techniques for plastic parts.

These forming techniques are all performed at a higher temperature. The polymer composition containing one or more metal ions and/or metal salts according to the present invention is thermostable and can therefore be used with polymers with a higher melting temperature, such as for instance PET and TPU polymers.

The polymer composition containing one or more metal ions and/or metal salts according to the present invention is of such a composition so that a homogeneous mixture can be obtained. In one embodiment, the mixture is mixed to obtain a homogeneous mixture. This is necessary, for example, with certain resins (such as polyurethane resins, epoxy resins, acrylic resins or silicone resins), where the monomers of the polymer composition are mixed with the metal ions and/or metal salts at room temperature and the whole is cured.

In one embodiment, the shape of the dental apparatus is adapted to the teeth of the wearer by means of a boil and bite technology, whereby the apparatus, by applying a warm temperature, can be partially melted and shaped and subsequently, by applying a cold temperature, hardened again into the desired shape.

In another preferred embodiment, the shape of the dental apparatus is adapted to the teeth of the wearer by heating the polymer in a heating machine and then applying a vacuum to it over an impression of the teeth.

In yet another embodiment, the shape of the dental apparatus is obtained by using a milling technology. In yet another embodiment, the shape of the dental apparatus is obtained by using a 3D printing technology. In yet another embodiment, the shape of the dental apparatus is obtained by (manually) molding a polymer composition. In a further embodiment, the polymer composition is first heated to a certain temperature before it is molded.

In what follows, the invention is described by way of non-limiting examples illustrating the invention, and which are not intended to and should not be interpreted as limiting the scope of the invention.

### EXAMPLES

### Example 1

To measure the release of zinc, a gingivitis mouthguard according to the present invention, where a concentration of approximately 0.081% zinc is contained in the polymer composition, was placed in a beaker and completely submerged in a known volume of a simulant. In the embodiment of Example 1, the polymer composition consists of 3% ethylene-vinyl acetate and 97% polypropylene, but it goes without saying that other polymer compositions are also possible. In this embodiment, zinc is present in the EVA polymer in the form of a mixture of zinc salts of certain fatty acids and the concentration of this zinc salt mixture in the EVA polymer is 2.7%. It goes without saying that other forms and concentrations of zinc may also be present in the polymer composition. In this example, water was used as simulant. The beaker was then placed in an oven at 70° C. for a total time of 2 hours. At the end of that time, the beaker was removed from the oven. The simulant was analyzed by ICP to determine the amount of zinc that may have migrated from the polymer. The migration of zinc from the polymer composition is 0.109 ppm when water was used as simulant. Migration tests with a gingivitis mouthguard made of a polymer composition comprising silver, gold, copper, platinum, tin or aluminum at a concentration of 0.081% also show limited migration of the respective metal ion from the polymer composition.

### Example 2

An orthodontic aligner made from an antimicrobial polymer composition according to the present invention, wherein a concentration of about 3% silver is contained in the polymer composition, was tested to evaluate the effectiveness of the orthodontic aligner against certain bacterial strains.

The product was tested for 2 types of bacterial strains (Escherichia Coli ATCC 8739 (Gram-) and Staphylococcus Aureus ATCC 6538 (Gram⁺)) using the international standard method for evaluating the antibacterial activity of non-porous plastic surfaces. The initial bacterial suspensions were diluted to obtain a certain bacterial concentration, expressed in colony forming units (cfu/ml). The orthodontic aligner and a control product without antibacterial properties were cut into optimally sized pieces for testing. The pieces of aligner and control product were treated with the aforementioned reference strains, covered with sterile polyethylene film and placed in an incubator at a temperature of about 37° C. for 24 hours. At the end of the incubation period, the samples were washed with a neutralizing solution, and the amount of bacteria was determined in this solution. The results obtained show that after 24 hours of incubation at 37° C., the amount of bacteria decreases in the group of the orthodontic aligner made of the aforementioned antibacterial polymer composition versus the control group. Notably, a decrease in colony forming units of 80% (in the case of Escherichia coli) and 93% (in the case of Staphylococcus aureus) was evident in the orthodontic aligner group versus the control group.

These tests were also repeated on the other polymer compositions according to the present invention and the results were consistent with those for the polymer composition described in Example 2.

### Example 3

A post-operative mouthguard made of an antimicrobial polymer composition according to the present invention was tested to evaluate its effectiveness in shortening the healing time of a surgical wound after placement of a dental implant. The polymer composition contained 0.25% copper and was applied to the wound for a minimum of 23 hours per day during the first 96 hours after surgery. The test was administered to a group of 10 patients who received an antimicrobial polymer mouthguard according to the present invention, 10 patients who received a non-antimicrobial mouthguard, and a final control group who did not receive a mouthguard. The findings of this test showed, when treated with the antimicrobial mouthguard of the present invention, a reduction in healing time of approximately 20% versus the control group without a mouthguard, and 15% versus the group receiving a non-antimicrobial mouthguard.

### Example 4

To measure the release of zinc from two different polymer compositions (“composition A⁺” and “composition B⁺”) according to the present invention, both polymer compositions with a concentration of about 0.01% zinc were completely immersed in a beaker with a known volume of water for a period of one week. Since saliva is largely composed of water, water has been deemed a suitable simulant for measuring this release.

Before and after this incubation period (at To and Ti, respectively), the concentration of zinc in the water was measured by ICP. As a control, both polymer compositions without zinc (“composition A⁻” and “composition B⁻”) were also included in the experiment. The concentration of zinc in both polymer compositions was also measured by ICP before and after the incubation period in the beaker with water (at To en Ti, respectively).

Table 1 below shows the average concentration of zinc measured in the water. This shows that the release of zinc from the polymer compositions after one week was very small, as the concentration of zinc in the water hardly increases.

This is also confirmed by the data in Table 2. This table shows the average concentration of zinc in the respective polymer compositions before and after the incubation period in water. This shows that the zinc concentration in the polymer compositions remains substantially unchanged after one week of incubation in the volume of water. These tests were also repeated on the other polymer compositions according to the present invention and the results were consistent with those for the polymer compositions described in the present example.

This data demonstrates the exceptionally low release of metal ions from the polymer compositions of the present invention.

TABLE 1

| | Concentration | |
|---|---|---|
| | Zn (mg/L) | SD |
| Water, T₀ | 0.010 | 0.001 |
| Water + Composition A⁺, T₁ | 0.051 | 0.013 |
| Water + Composition B⁺, T₁ | 0.017 | 0.005 |
| Water + Composition A⁻, T₁ | 0.026 | 0.002 |
| Water + Composition B⁻, T₁ | 0.012 | 0.001 |

Determination of Zn concentration (mg/L) in a beaker with a known volume of water by ICP. Mean values from three measurements and the corresponding standard deviations (SD) are shown for measurements before and after a one-week incubation period (at T₀ and T₁, respectively) with composition A with or without 0.01% zinc (composition A⁺ and A⁻, respectively) or with composition B with or without 0.01% zinc (composition B⁺ and B⁻, respectively).

TABLE 2

| | concentration | |
|---|---|---|
| | Zn (mg/kg) | SD |
| Composition A⁺, T₀ | 664.0 | 15.0 |
| Composition A⁺, T₁ | 657.0 | 16.1 |
| Composition A⁻, T₀ | 1.4 | 1.0 |
| Composition A⁻, T₁ | 1.0 | 0.9 |
| Composition B⁺, T₀ | 593.0 | 5.6 |
| Composition B⁺, T₁ | 599.3 | 5.9 |
| Composition B⁻, T₀ | 13.2 | 0.65 |
| Composition B⁻, T₁ | 11.9 | 0.64 |

Determination of Zn concentration (mg/kg) by ICP of different polymer compositions before and after a one-week incubation period (T₀ and T₁, respectively) in a beaker with a known volume of water. “⁺” denotes a composition containing Zn at a concentration of 0.01%. “⁻” denotes a composition to which no Zn has been added. The mean of three measurements and the corresponding standard deviation is shown in the table.

1. A dental apparatus, wherein the apparatus is made of a polymer composition having antimicrobial activity, wherein the polymer composition comprises one or more metal ions and/or metal salts at a concentration of between 0.001% and 10%.

2. The dental apparatus according to claim 1, wherein the released concentration of the metal ions when using the apparatus in the oral cavity of a patient is up to a maximum of 21 ppm.

3. The dental apparatus according to claim 1, wherein the released concentration of the metal ions when using the apparatus in the oral cavity of a patient is between 0 and 10 ppm.

4. The dental apparatus according to claim 1, characterized in that the metal ions and/or salts of said metal ions are dispersed or dissolved in the polymer composition.

5. The dental apparatus according to claim 1, wherein the metal ion is selected from the group consisting of Al (aluminum), Si (silicon), Ti (titanium), V (vanadium), Cr (chromium), Mn (manganese), Co (cobalt), Ni (nickel), Cu (copper), Zn (zinc), Zr (zirconium), Nb (niobium), Mo (molybdenum), Pd (palladium), Ag (silver), Sn (tin), Se (selenium), Ta (tantalum), W (tungsten), Pb (lead), Au (gold), Pt (platinum) or a combination of these.

6. The dental apparatus according to claim 1, wherein the metal ion is zinc and wherein zinc is present in the polymer composition in the form of a zinc salt.

7. The dental apparatus according to claim 1, wherein the polymer composition consists of one or more polymers selected from acrylates and methacrylates, polyesters, thermoplastic polyurethanes (TPU), polyether blockamides (PEBAX), polyolefins and polyolefin copolymers, styrene-based thermoplastics, polyamides, polycarbonate (PC) and polyacetals, silicones or a mixture thereof.

8. The dental apparatus according to claim 1, wherein the apparatus comprises orthodontic braces, a sports mouthguard, a gingivitis mouthguard, an orthodontic aligner, a temporary crown, a permanent crown, a bite plate, dentures, an antibacterial pad, a palatal splint, an anti-snoring device, a whitening shield, a bruxism splint, a coping, a fluoride shield, an individual impression tray, a Michigan splint, a medication shield, a radiation shielding splint, an occlusal splint, an interim prosthesis, or a similar device.

9. The dental apparatus according to claim 1 for use in the prevention or treatment of bacterial oral diseases.

10. A method of producing a dental apparatus according to claim 1, wherein one or more metal ions and/or metal salts are mixed with a polymer before the dental apparatus is formed by injection molding, extrusion, thermoforming, compression molding or 3D printing of the mixture.

11. The method according to claim 10, comprising
a. dissolving or dispersing the one or more metal ions and/or metal salts in an aqueous or organic solvent thereby forming an obtained solution or dispersion of the metal ion and/or metal salt,
b. adding the obtained solution or dispersion of the metal ion and/or metal salt to a solution or a dispersed phase of monomers suitable for synthesizing a polymer by means of a polymerization reaction,
c. carrying out the polymerization reaction,
d. forming a dental apparatus by the injection molding, extrusion, thermoforming, compression molding or 3D printing of the polymerized composition.

12. The method according to claim 11, wherein step b takes place before or simultaneously with step c.

13. The dental apparatus according to claim 7, wherein the acrylates and methacrylates are chosen from polymethyl acrylate (PMA) or polymethyl methacrylate (PMMA));
wherein the polyesters are chosen from polyethylene terephthalate (PET), polyethylene terephthalate glycol (PETG), polycaprolactone (PCL), polysulfone (PSU), polylactic acid (PLA), poly(lactic-co-glycolic acid) (PLGA), polybutylene terephthalate (PBT), polyethylene furanoate (PEF), polypropylene terephthalate (PPT), polybutylene adipate terephthalate (PBAT), polybutylene succinate (PBS), or polybutylene succinate adipate (PBSA);
wherein the polyolefins and polyolefin copolymers are chosen from polyethylene (PE), polypropylene (PP), ethylene vinyl acetate (EVA), polyvinyl butyraldehyde (PVB), polybutylene (PB), polyisobutylene (PIB), or ethylene propylene diene monomer (EPDM);
wherein the styrene-based thermoplastics are chosen from poly(styrene-butadiene-styrene) (SBS), polystyrene, acrylonitrile butadiene styrene (ABS), acrylonitrile styrene acrylate (ASA), or styrene acrylonitrile (SAN);
wherein the polyamides are chosen from polyamide 6, polyamide 6,6 or polyamide 12; and
wherein the polyacetals are polyoxymethylene (POM).

14. The dental apparatus according to claim 9, wherein the bacterial oral disease is gingivitis, periodontitis or halitosis.

15. The dental apparatus according to claim 6, wherein the zinc salt is zinc PCA, zinc oxide, zinc hydroxide, zinc pyrrolidone, zinc pyrithione, a zinc salt of a fatty acid or a mixture thereof.

16. The dental apparatus according to claim 3, wherein the released concentration of the metal ions is between 0 and 5 ppm.

17. The dental apparatus according to claim 16, wherein the released concentration of the metal ions is between 0 and 1 ppm.

18. The dental apparatus according to claim 17, wherein the released concentration of the metal ions is between 0 and 0.5 ppm.
